(19) United States
(12) Patent Application Publication
(10) Pub. No.: US 2004/0091521 A1
(43) Pub. Date: May 13, 2004

(54) Multilayer backing construction for active substance patch systems

(75) Inventors: Detlev Radloff, Hamburg (DE); Andreas Schabert, Seevetal (DE); Matthias Wasner, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/436,680

(22) Filed: May 12, 2003

Related U.S. Application Data
(63) Continuation of application No. PCT/EP01/12602, filed on Oct. 31, 2001.

(30) Foreign Application Priority Data
Nov. 11, 2000 (DE) 100 56 014.8

(51) Int. Cl. A61K 9/70; A61F 13/00

(52) U.S. Cl. 424/449

(57) ABSTRACT

An active substance patch comprising a laminate coated on its skin-facing side with an adhesive comprising an active substance. In one embodiment, the laminate has at least two layers. More particularly, in this embodiment, the side of the laminate remote from the skin includes a barrier layer that is impermeable to the active substance, and the skin-facing side of the laminate includes a backing layer. These two layers are preferably configured so that they can be separated from one another by a user.

### CROSS-REFERENCE TO RELATED APPLICATIONS

This is a continuation application of PCT/EPO1/12602, filed Oct. 31, 2001, which is incorporated herein by reference in its entirety, and also claims the benefit of German Priority Application No. 100 56 014.8, filed Nov. 11, 2000.

### FIELD OF THE INVENTION

The present invention relates to the embodiment of a backing material for active substance patch systems, generally referred to as Transdermal Therapeutic Systems (TTS).

### BACKGROUND OF THE INVENTION

Transdermal Therapeutic Systems (TTS) for delivering active substances through the skin have been known for a long time. The topical application of drugs by way of active substance patch systems offers two main advantages. First, this form of administration produces first-order release kinetics of the active substance, thereby enabling a constant level of active substance to be maintained in the body over a very long period. Second, the path of uptake through the skin avoids the gastrointestinal tract and also the first liver passage. As a result, selected drugs may be effectively administered in a low dose. This is particularly advantageous when the drug is desired to act locally while avoiding a systemic effect. This is the case, for example, with the treatment of rheumatic joint complaints or muscular inflammation.

One embodiment of such transdermal systems which has been well described in the technical literature is that of matrix systems or monolithic systems, in which the drug is incorporated directly into the pressure sensitive adhesive. In the ready-to-apply product, a pressure sensitively adhesive matrix comprising active substance of this kind is equipped on one side with a release film given an anti-adhesive finish, which is removed prior to application to the skin. On the opposite side there is a backing film which constitutes a definite constituent of the system and remains of the patch during application.

Highly specific requirements are imposed on this backing film of a transdermal therapeutic system. For example, depending on the size of the patch applied, the material used must preferably posses sufficient flexibility and elasticity to ensure adequate patient comfort. If the backing film used is too rigid the patient will be uncomfortable. Additionally, when applied to moving areas of the body, an insufficiently elastic backing material may result in detachment of parts of the product or even of the entire product. That would prevent the transport of active substance through the skin and jeopardize the effectiveness of the TTS.

On the other hand, one function of the backing layer is to reliably prevent loss of active substance over the period of storage. The period of storage is the time between manufacture of the product and its application to the patient. The maximum time frame is frequently defined by way of the maximum shelf life, which generally encompasses three years. From this long period of time it is clear that the material used must constitute a very effective barrier both to the active substance used and to the auxiliaries employed.

The problem surrounding these two requirements is in particular that existing barrier materials are of poor flexibility and elasticity. Known flexible and elastic backing materials, on the other hand, are generally characterized by a very low barrier effect with respect to migratable molecules.

Backing materials for bandage systems play a particularly important function in wound care. In these applications, the focus is on the wear comfort of the patient, the duty of care applying in particular to injuries to high-movement joints such as in the knee and elbow regions, for example, or on the hand. The materials employed in this context have in the past frequently been very soft PVC films, which have slowly been replaced by polyolefin films. Modern products are frequently equipped with a non-woven backing.

EP 0 749 756 A2 describes for example a non-woven material based on polyester elastomers as a backing material for a bandage for wound care. Owing to the excellent elasticity and conformability of this material, a high degree of wear comfort is achieved. It is further increased by the high water vapor permeability of the backing described. Nonwovens, however, have a microporous structure which contradicts an effective barrier action. Migratable ingredients of a patch system can be volatilized very rapidly through such materials. For this reason, such a system is unsuitable for use in the field of active substance patches.

A very good barrier effect is possessed in particular by films of polyethylene terephthalate (PET). Consequently, this material is widespread in the packing industry in the sector of flavor tight and gas tight packaging. In the area of Transdermal Therapeutic Systems, as well, PET is employed with great frequency. Virtually all of the release films used here consist of PET.

The reason for this lies not only in the outstanding barrier effect of PET but also in its extreme mechanical stability. This leads to distinct advantages during the production of these systems, here in particular in the course of coating and converting, by punching for example. Flexible materials are substantially more difficult to deal with in these operating steps.

As a backing material, PET is unsuitable owing to its low flexibility and elasticity, despite being very widespread as such in the absence of suitable alternatives. Another reason for this is that conventional active substance patches can be kept very small in terms of their dimensions. In the case of drugs with a systemic action, moreover, the site of dermal application is relatively unimportant, and so the patch can be applied in the area of body regions where there is very little movement. The chest area in particular may be mentioned here.

The described problem of flexible backing materials having good barrier properties is known in the literature, and numerous solutions are given.

DE 195 46 024 A1 applies itself to the problem mentioned. The disadvantage of a deficient barrier effect on the part of flexible backing films is profitably exploited here. Thus, a backing system which is permeable to the active substance is praised as an additional active substance reservoir, allowing a substantial reduction in the overall thickness of the patch system. The reduced thickness achieved in this way raises the flexibility of the overall product and so enhances the desired patient comfort. However, the problem of the loss of active substance which occurs remains unresolved.

A compromise between wear comfort and ease of processing is described by WO 99/12529 A1. There, a unidirectionally elastic backing is used. As a result, it is possible to use a material which is rigid in the processing direction, producing substantial advantages during the manufacturing operation. Because of the elasticity perpendicular to this direction, however, an acceptable wear comfort is achieved. The problem of the barrier effect, however, is not mentioned.

A very supple patch is described by WO 98/29143 A1. In that case, a backing material is employed which following application to the skin is removed. In the cited document the backing material to be removed is referred to as a “supporting layer”. The underlying pressure-sensitively adhesive layer is given an anti-adhesive finish in order to prevent sticking to the clothing. Achieved as a result is an extremely thin and therefore highly flexible product construction.

Distinct disadvantages of this design, however, exist in respect of the ease or processing of such a system and also the ease of handling for the patient. The absence of a backing during application makes it significantly more difficult to detach the patch following use. Adhesives used generally do not possess the necessary cohesiveness to allow the bandage to be removed in one piece after the period of wear. Additionally, in the course of wear, the patch is highly sensitive to mechanical stresses, for example, the friction which occurs with the clothing. Moreover, no solution is provided as to how the necessary supporting film adheres to a non-adhesively configured layer.

### SUMMARY OF THE INVENTION

It is an object of one embodiment of the invention to provide an active substance patch whose backing exhibits a sufficient barrier effect and which avoids the disadvantages known from the prior art. The material should preferably be able to be manufactured inexpensively and should preferably be ecologically unobjectionable, while also offering pleasant wear comfort in use and harmonizing the stated basic requirements of the backing of a TTS.

### BRIEF DESCRIPTION OF THE DRAWINGS

FIG. 1 is a graph of ibuprofen permeability of various film materials as a function of the elasticity modulus of the materials.

FIG. 2 is a graph of oxygen permeability of various film materials as a function of the elasticity modulus of the materials.

### DETAILED DESCRIPTION OF THE INVENTION

The invention accordingly provides an active substance patch comprising a laminate coated on its skin-facing side with an adhesive which comprises an active substance, the laminate having at least two plies.

The side of the patch remote from the skin carries a barrier layer which is impervious to the active substance, while its skin-facing side carries a backing layer, and these two layers can be separated from one another.

The side facing the active substance-containing adhesive is composed ideally of a conformable elastic material, while the side remote from the adhesive is composed of a barrier layer. The connection between the two layers is on the one hand firm enough to remain stable during processing and storage while on the other hand sufficiently labile to be separated before or shortly after application to the skin. Accordingly, the barrier layer, which is no longer needed during application, is removed, and the flexible backing remains on the patch.

In contrast to the unbacked product described in WO 98/29143 the product of the invention is protected very effectively against external influences. For example, depending on the corresponding embodiment, bathing or showering during application is possible. The flexible backing film which remains also ensures easy reattachment in one piece.

For the construction of a laminate in accordance with the invention any materials known in this respect can be used for the barrier layer.

Such materials include in particular polymer films which owing to their microstructure have a good barrier effect for aromas, and also films based on partially crystalline polymers such as for example polyethylene terephthalate and/or its derivatives and also polyamides and polyimides, and films based on halogenated polymers, polyvinyl chloride (PVC) and polyvinylidene chloride (PVDC) for example.

In one preferred embodiment, accordingly, the barrier layer comprises films based on polyamide, polyimide, polyethylene terephthalate and/or its derivatives or halogenated polymers.

With further preference the barrier layer is applied to the backing layer by a chemical or physical process.

Embraced by this in particular are polymer films and papers which are modified by metal vapor deposition or metal coating—with aluminum, for example—or by a coating of metal oxides such as SiOₓ or AlOₓ for example.

The backing layer preferably comprises polyolefin films based on ethylene or propylene, synthesized either conventionally or by metallocene catalysis, nonwovens or knits, composed in particular of polyester, polyamides, viscose or cotton, and also laminates thereof.

For the adhesive coating it is preferred to use commercially customary pressure sensitive adhesives based on acrylate or rubber.

The application weight of the adhesive on the patch lies in particular in the range from 100 to 500 g/m², more preferably 300 g/m².

In another preferred version of the invention the adhesive is composed of a pressure-sensitive adhesive matrix containing active substances if desired.

By way of example, the matrix can be free from mineral oils and tackifies resins and may comprise the following constituents:

a) synthetic framework polymers based on polyisobutylene at from 25 to 90% by weight,

b) amorphous poly-α-olefins at from 5 to 40% by weight,

c) at least one insoluble hydrophilic filler having an average particle size of less than 100 μm at from 10 to 60% by weight, and

d) if desired, a drug at from 0.001 to 20% by weight.

In a first advantageous embodiment the polyisobutylene is composed of high molecular mass PIB at from 5 to 30% by weight and low molecular mass PIB at from 20 to 60% by weight.

A typical pressure sensitive adhesive of the invention is therefore composed of the following components:

| | | |
|---|---|---|
| High molecular mass PIB | 5-30% | by weight, preferably |
| | 10-20% | by weight |
| Low molecular mass PIB | 20-60% | by weight, preferably |
| | 30-50% | by weight |
| Amorphous poly-α-olefin | 5-30% | by weight, preferably |
| | 5-20% | by weight |
| Hydrophilic filler | 20-60% | by weight, preferably |
| | 30-50% | by weight |
| Optional drug | 0.001-20% | by weight, preferably |
| | 1.0-5.0% | by weight |

As an option it is also possible to add up to 20% by weight of a permeation promoter.

The stated formula ingredients in one embodiment of the invention are defined more precisely as follows:

### High Molecular Mass PIB

Polyisobutylene having a weight-average molecular weight (M_{w}) of from 500,000 to 1,100,000, preferably between 650,000 and 850,000. Such polymers are available commercially for example under the trade names Oppanol B100 (BASF) or Vistanex MM-L80 (Exxon).

### Low Molecular Mass PIB

Polyisobutylene having a weight-average molecular weight (M_{w}) of from 40,000 to 120,000, preferably between 60,000 and 100,000. Such polymers are available commercially for example under the trade names Oppanol B15 (BASF) or Vistanex LMMH (Exxon).

### Amorphous poly-α-olefin

Amorphous copolymers based on ethylene and propylene, butylene or 1-hexene. The preferred weight-average molecular weight (M_{w}) is from 5,000 to 100,000, more preferably between 10,000 and 30,000. Such polymers are available commercially for example under the trade names Eastoflex® (Eastman) or Vestoplast® (Hüls).

### Hydrophilic Filler

Hydrophilic particles insoluble in the stated polymer matrix and based on cellulose. Preference is given to an average particle size of less than or equal to 100 μm with as uniform as possible a surface. Such materials are available commercially for example under the trade names Avicel (FMC) and Elcema (Degussa-Hüls).

Preparation takes place preferably in a process in which all of the components are homogenized in the melt with no solvent being added.

Particular preference is given to processing all of the components in a continuous or batchwise operation at a temperature below 100° C.

The adhesive is distinguished by outstanding adhesion properties to the skin, by easy and painless redetachability, and in particular by its extremely low potential to induce skin irritation. The preparation operation proceeds with the complete omission of solvents.

Without making any claim to completeness, typical active substances in the adhesive in the context of the present invention include the following:

| Indication: | Active substance |
|---|---|
| Antimycotics | naftifine |
| | amorolfine |
| | tolnaftate |
| | ciclopirox |
| Antiseptics | thymol |
| | eugenol |
| | triclosan |
| | hexachlorophene |
| | benzalkonium chloride |
| | clioquinol |
| | quinolinol |
| | undecenoic acid |
| | ethacridine |
| | chlorhexidine |
| | hexetidine |
| | dodicine |
| | iodine |
| Nonsteroidal antirheumatics | glycol salicylate |
| | flufenaminic acid |
| | etofenamat |
| | ketoprofen |
| | piroxicam |
| | indomethacin |
| Antipruritics | polidocanol |
| | isoprenaline |
| | crotamiton |
| Local anesthetics | benzocaine |
| Antipsoriatics | ammonium bitumasulfonate |
| Keratolytics | Urea |

Mention may also be made of hyperemic active substances such as natural active substances of Cayenne pepper or synthetic active substances such as nonivamide, nicotinic acid derivatives, preferably benzyl nicotinate or propyl nicotinate.

If desired, the open, adhesive side of the patch, the side to be applied to the skin, can be lined with a redetachable protective liner layer. It is additionally possible to dispose a customary wound contact material on the self-adhesive coating.

The patch of the invention is outstandingly suitable for meeting the two main requirements imposed on the backing system, at different times.

During the period of storage of the patch, it is important to ensure a very good barrier effect by the backing. Elasticity and flexibility are not necessary at this point in time. Instead, the rigidity of an effective barrier layer during the storage period results in additional protection against mechanical stress such as during transit.

When the product is subsequently applied to the patient, the barrier effect of the backing layer is not needed. In comparison with the storage time, the period of application, which is up to 24 hours, is minimal. The flexibility and elasticity of the backing material are important during application.

The inventive embodiment of a patch reliably prevents the migration of the active substance from the patch system over the period ranging from storage until application to the skin. In the course of application on the skin the system of the invention, owing to its high flexibility and elasticity, leads to a pleasant wear sensation. This ensures a high wear comfort for the patient and a long-term stability of the active substance content.

Particularly advantageous embodiments of the patch will be described below with reference to a number of examples, without thereby wishing to subject the invention to any unnecessary restriction.

### EXAMPLES

Combinations of various materials are suitable for the construction of the backing system in accordance with the invention, and an extract of such combinations will be cited here as examples. The modulus of elasticity (elasticity modulus) serves as a measure of the elasticity of the film materials. A high elasticity modulus indicates low elasticity or flexibility and hence indicates low wear comfort. A low elasticity modulus indicates a material of high elasticity and flexibility and hence indicates high wear comfort. Besides the oxygen permeability the nonsteroidal active substance ibuprofen is employed as an example substance for the permeability of backing materials.

| | | Ibuprofen | | |
|---|---|---|---|---|
| | | permeation rate** | | Oxygen |
| | Elasticity | [mg/(d cm²)] @ | | permeability*** |
| | modulus* | 40° C. various | | @ 20° C., 65% RH. |
| Material | [MPa] @ RT | film thicknesses | | [cm³/(m² d atm)] |
| PET | 3100 | 12 μm | 0.013 | 50 |
| Polyamide 6 | 2400 | 81 μm | 0.077 | 35 |
| PP | 1700 | 72 μm | 3.0 | 3000 |
| LDPE | 310 | 90 μm | 47.7 | 10000 |
| LDPE | 310 | 75 μm | 50.8 | — |
| EVA | 59 | 25 μm | 56.2 | 18000 |

*Irvin I. Rubin: Handbook of Plastic Materials and Technology, John Wiley & Sons, New York, 1990
**Fraunhofer-Institut f\ddot{ur} Verfahrenstechnik und Verpackung IVV: order number PA/4146/99, report of Apr. 28, 2000.
***J. Nentwig: Kunststoff-Folien, Carl Hanser Verlag, Munich Vienna, 1994; film thickness 20 μm

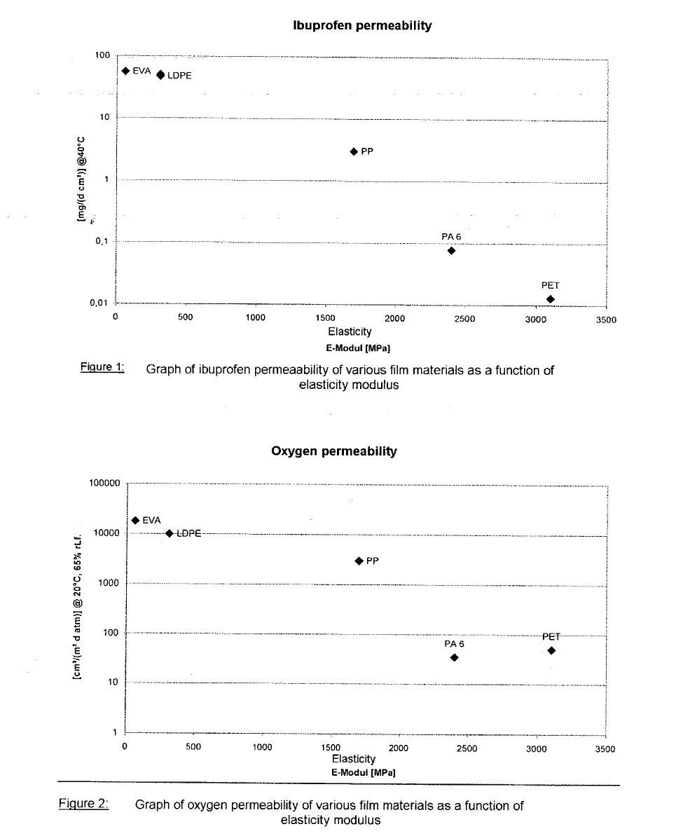

The examples illustrate a marked correlation between active substance permeability and elasticity: the higher the modulus of elasticity, the lower the active substance permeability. This trend reoccurs for the oxygen permeability, which is frequently used as a measure of aroma permeability. FIGS. 1 and 2 illustrate these correlations.

### Example 1

Laminate in the combination of PET as backing material with a high barrier property and a low elasticity modulus and LDPE as a flexible film with a low barrier effect against active substance migration (see FIGS. 1 and 2).

### Example 2

Laminate in the combination of PET as backing material with a high barrier property and a low elasticity modulus and EVA as a flexible film with a low barrier effect against active substance migration (see FIGS. 1 and 2).

### Example 3

Laminate in the combination of PA 6 as backing material with a high barrier property and a low elasticity modulus and PP as a flexible film with a low barrier effect against active substance migration (see FIGS. 1 and 2).

### Example 4

Laminate in the combination of PA 6 as backing material with a high barrier property and a low elasticity modulus and LDPE as a flexible film with a low barrier effect against active substance migration (see FIGS. 1 and 2).

What is claimed is:

1. An active substance patch comprising:
a laminate comprising: (A) a skin-facing backing layer, and (B) a barrier layer that is configured to be remote from a user's skin when said patch is in use, said barrier layer being impermeable to an active substance; and
an adhesive comprising said active substance, said adhesive being disposed on a skin-facing portion of said skin-facing backing layer,
wherein said barrier layer is configured so that it may be separated from said backing layer by a user.

2. The active substance patch of claim 1, wherein said barrier layer comprises a film based on polyamide.

3. The active substance patch of claim 2, wherein said backing layer comprises a polyolefin film.

4. The active substance patch of claim 1, wherein said barrier layer comprises a film based on a derivative of polyamide.

5. The active substance patch of claim 1, wherein said barrier layer comprises a film based on a hologenated polymer of polyamide.

6. The active substance patch of claim 1, wherein said barrier layer comprises a film based on polyimide.

7. The active substance patch of claim 1, wherein said barrier layer comprises a film based on a derivative of polyimide.

8. The active substance patch of claim 1, wherein said barrier layer comprises a film based on a hologenated polymer of polyamide.

9. The active substance patch of claim 1, wherein said barrier layer comprises a film based on polyethylene terephthalate.

10. The active substance patch of claim 1, wherein said barrier layer comprises a film based on a derivative of polyethylene terephthalate.

11. The active substance patch of claim 1, wherein said barrier layer comprises a film based on a hologenated polymer of polyethylene terephthalate.

12. The active substance patch of claim 1, wherein said barrier layer is applied to said backing layer by a chemical process.

13. The active substance patch of claim 1, wherein said barrier layer is applied to said backing layer by a physical process.

14. The active substance patch of claim 1, wherein said backing layer comprises a polyolefin film.

15. The active substance patch of claim 14, wherein said polyolefin film is based on ethylene or propylene.

16. The active substance patch of claim 1, wherein said backing layer comprises a nonwoven.

17. The active substance patch of claim 16, wherein said nonwoven comprises one or more materials selected from the group consisting of: polyester, one or more polyamides, viscose and cotton.

18. The active substance patch of claim 1, wherein said backing layer comprises a knit.

19. The active substance patch of claim 18, wherein said knit comprises one or more materials selected from the group consisting of: polyester, one or more polyamides, viscose and cotton.

20. An active substance patch comprising:
a backing layer having both a first side, and a second side that is opposite said first side;
a barrier layer disposed adjacent said first side of said backing layer, said barrier layer being impermeable to an active substance; and
an adhesive disposed adjacent said second side of said backing layer, said adhesive comprising said active substance,
wherein said barrier layer is configured so that it may be separated from said backing layer by a user.

21. A method of applying an active substance patch comprising:
using an adhesive disposed on said patch to affix said patch to a user, said adhesive comprising an active substance; and
removing a barrier layer from said patch, said barrier layer being substantially impermeable to said active substance.

22. The method of claim 21, wherein said step of removing said barrier layer is performed before said step of affixing said patch to said user.

23. The method of claim 21, wherein said step of removing said barrier layer is performed after said step of affixing said patch to said user.

24. The method of claim 21, wherein said patch comprises a backing layer disposed between said barrier layer and said adhesive.
